(19) United States
(12) Patent Application Publication
(10) Pub. No.: US 2009/0087859 A1
(43) Pub. Date: Apr. 2, 2009

(54) Microfluidic device for detecting soluble molecules

(76) Inventor: Brandon T. Johnson, Allston, MA (US)

Correspondence Address: CLARK & ELBING LLP, 101 FEDERAL STREET, BOSTON, MA, 02110, US

(21) Appl. No.: 11/230,426

(22) Filed: Sep. 20, 2005

Related U.S. Application Data
(60) Provisional application No. 60/611,475, filed on Sep. 20, 2004.

(51) Int. Cl. G01N 33/53; C12M 1/34

(52) U.S. Cl. 435/7.1; 435/288.7; 435/287.2

(57) ABSTRACT

The present disclosure provides a microfluidic device that is compatible with standard centrifuges and may be used for point-of-care disease detection. The detection methodology may be based on microELISA.

### CROSS-REFERENCE TO RELATED APPLICATIONS

This application claims benefit of U.S. Provisional Application 60/611,475, filed Sep. 20, 2004.

### FIELD OF THE INVENTION

This invention relates to microfluidic devices and their use in the detection of disease.

### BACKGROUND OF THE INVENTION

Sexually transmitted diseases remain a public health issue in many communities. According to the American Social Health Association, a partner of the Center for Disease Control and Prevention, 65 million Americans are currently infected with a sexually transmitted disease (STD) and 15 million are infected each year. Most STDs are curable, and early detection can prevent complications and the continued spread of the disease. Treatment and counseling for these diseases are available, but the inability to perform testing at the point-of-care delays results and hinders the effectiveness of public health workers to stem these epidemics.

Several problems exist with the testing methods currently available in clinical settings. Many tests cannot be performed at point-of-care facilities and results can take weeks to obtain. For STD testing, these problems are compounded by social stigma and anxiety and convincing patients to make a return visit can be problematic. Current methods often require large blood samples, limiting the number of diseases for which an individual can be tested per visit. Other tests require uncomfortable sample collection methods. For example, Chlamydia testing in men requires specimens collected from inside the urethra with a cotton swab. While less unpleasant testing methods are available, this method is widely used because it is fast and cost effective.

Delayed results, availability problems, uncomfortable sample collection and the cost of these tests all impede their use at point-of-care facilities, making it easy to understand why less than half of American adults between ages 18 and 44 have been tested for non-HIV/AIDS STDs. STDs are more prevalent in low income areas where patients may not be able to afford testing, but even well funded facilities cannot afford to run the most sophisticated tests, like polymerase chain reaction (PCR) detection, for every patient in every case. Many point-of-care facilities do not have the space or money to dedicate to the equipment required for these tests. In these cases, doctors will diagnose their patients by outward symptoms only and sometimes prescribe antibiotics unnecessarily because testing is simply not feasible. Many STDs can cause severe damage and be spread without ever exhibiting symptoms. Chlamydia, for example, only expresses symptoms about 25% of the time increasing the risk of unknowingly transmitting the disease. Chlamydia and gonorrhea are the main causes of pelvic inflammatory disease, the leading cause of sterility among women.

There is a need in the medical community for rapid and inexpensive detection of STDs and other diseases. Further, it is preferable if these detection methodologies can be delivered at the point-of-care in a single patient visit.

U.S. Pat. Nos. 6,063,589 and 6,527,432 disclose microfluidic devices on “spinning disks” that are substantially two-dimensional. The “spinning disks” may contain multiple detection devices; however, each device may have to be individually filled with the biological sample.

U.S. Pat. No. 6,479,239 discloses a device for detecting and identifying infectious disease agents using physical separation techniques. Ultracentrifuge tubes having successively smaller diameters are used to characterize infectious agents based on their size. Secondary detection methods may be used for additional characterization.

U.S. Pat. No. 6,551,841 discloses a microfluidic device capable of detecting soluble analytes. The sample fluid is forced through the device using a pump or by capillary action.

U.S. Pat. No. 6,929,239 discloses a microfluidic device, in a “card” configuration capable of conducting multiple simultaneous chemical reactions utilizing an internal network of interconnecting ducts and channels.

### SUMMARY OF THE INVENTION

The present disclosure provides a microfluidic-based device for the detection of disease (e.g., infectious disease agents) that is inexpensive to manufacture and operate and provides rapid results. Ideally, the device disclosed herein may be configured for use in conjunction with standard, low cost centrifuges, including basic clinical centrifuges normally used in the preparation of blood samples at point-of-care facilities.

In one aspect, the invention provides a non-radial cylindrical microfluidic device for analyzing the presence or absence of a molecule to be detected in a fluid sample. The device comprises a sample reservoir having a sample input port in fluid connection with at least one detector array, wherein each detector array comprises at least one detector that comprises a reaction chamber comprising an immobilized capture molecule, and a reagent capable of undergoing a colorimetric reaction or displaying an optically detectable signal and capable of reacting with the molecule to be detected.

The device of this invention may be used to assess the presence or absence of a molecule is biological fluids such as whole blood, blood serum, blood plasma, seminal fluid, prostatic fluid, saliva, urine, and spinal fluid.

The fluid sample may be moved from the sample reservoir to the reaction chamber by any appropriate means including, for example, passively by capillary action, or actively by centripetal force arising from centrifugation of the device or by gas pressure applied to the sample reservoir. Further, all fluids may be moved through the device, ideally using centripetal force arising from centrifugation of the device, or by gas pressure.

In one embodiment, the detector may further comprise a diluent chamber and diluent, and a mixing channel capable of generating turbulent flow, wherein the mixing channel is upstream from the reaction chamber. The fluid channel leaving the diluent chamber joins the fluid channel leaving the sample reservoir upstream from the mixing channel. In a related embodiment, the diluent chamber is isolated from the mixing channel by a first burst valve.

In another embodiment, the detector(s) contain a sample chamber having a pre-defined capacity. In a related embodiment, the loading channels connecting the sample reservoir to the sample chambers is sloped down in the direction of the sample reservoir such that, under centrifugation, excess sample fluid not accommodated by the sample chambers flows back into the sample reservoir.

In preferred embodiments, the reagent that is capable of undergoing a calorimetric reaction or displaying an optically detectable signal is further capable of binding to the molecule to be detected.

In one embodiment, the immobilized capture molecule (immobilized within the reaction chamber) is an antigen, the molecule to be detected is a serum antibody or other soluble binding protein, and the reagent is a detectably labeled antibody capable of binding to the serum antibodies or other soluble binding protein contained in the fluid sample. The detectably labeled antibody may be an enzyme-linked antibody or an antibody having a fluorescent or other optically readable tag. The calorimetric reaction and/or optically readable tag may be qualitatively assessed by the operator or may be qualitatively assessed or measured by an optical sensing device (e.g., a detector).

Although any antigen may be used in this embodiment, useful antigens include those that are specific for microorganisms that cause sexually transmitted diseases including, for example, Chlamydia spp., Gonorrhea spp., human Papillomavirus, herpes simplex virus, hepatitis B, syphilis, trichomononiasis, bacterial vaginosis, and human immunodeficiency virus.

In another embodiment, the immobilized capture molecule is an antibody specific for a pre-determined antigen. Although an antibody specific for almost any antigenic molecule may be used, particularly useful capture antibodies include those that bind to microorganism-specific antigens, such as antigens specifically associated with Chlamydia spp., Gonorrhea spp., human Papillomavirus, herpes simplex virus, hepatitis B, syphilis, trichomononiasis, bacterial vaginosis, and human immunodeficiency virus.

In this embodiment, the reagent is a detectably labeled antibody capable of binding to the antigen to be measured contained in the fluid sample. The detectably labeled antibody may be an enzyme-linked antibody or an antibody having a fluorescent or other optically readable tag. The calorimetric reaction and/or optically readable tag may be qualitatively assessed by the operator or may be qualitatively assessed or measured by an optical sensing device (e.g., a detector) using well known detection techniques.

In another embodiment, the immobilized capture molecule is an oligonucleotide, the molecule to be detected is a polynucleic acid (e.g., DNA or RNA), and the reagent is a detectably labeled oligonucleotide.

In another embodiment, the device further contains a wash chamber containing a wash buffer and a developing chamber containing the reagents, wherein the wash chamber is isolated from the reaction chamber by a second burst valve and the developing chamber is isolated from the reaction chamber by a third burst valve, wherein the first burst valve is designed to rupture at a substantially lower pressure than the second burst valve and the second burst valve is designed to rupture at substantially lower pressure than the third burst valve. The pressure differences that cause the first, second, and third burst valves to rupture may be advantageously produced by different centrifugal forces. Specifically, the operator may control the centrifugal force applied to the device by controlling the revolution speed of the centrifuge.

In a related aspect, the invention provides a method for detecting a molecule in a fluid sample comprising introducing the sample into a device of any of the foregoing aspects and embodiments, centrifuging the device, detecting the presence or absence of the calorimetric response, and relating the presence or absence of the calorimetric response to the presence or absence of the molecule to be detected.

It is contemplated that the devices disclosed herein are microfluidic devices having channel diameters of 10-1000 μm, preferably 100-500 μm, and chamber capacities of 1-1000 μl, preferably 10-500 μl. It is well recognized that higher centrifuge speeds (i.e., centrifugal forces) are required as channel and chamber sizes are reduced. In preferred embodiments, the devices of the invention are designed to run on standard clinical blood centrifuges operating at 500-1500 rpm.

In another aspect, the invention provides a diagnostic kit for use in detecting a molecule associated with a disease state. The kit comprises any of the devices described above along with instructions for its use.

“Non-radial cylindrical device,” as used when referring to devices of the present invention, means a device having a length that is greater than its radius. Non-radial cylindrical devices are designed to be revolved, such as in a centrifuge, rather than rotated around a central axis such as is the case for a “spinning disk” device. One distinguishing feature of a non-radial cylindrical device is that the direction of the force vector during centrifugation is constant over the entire device. By contrast, force vectors on “spinning disk” devices radiate outward in all directions simultaneously from the center of the disk.

### BRIEF DESCRIPTION OF THE DRAWINGS

Further understanding of the principles of the present invention may be had by reference to the accompanying drawings, wherein:

FIG. 1 is a perspective view of a microfluidic device in accordance with the principles of this disclosure;

FIGS. 2A-C are schematic diagrams of microfluidic devices in accordance with the principles of this disclosure. FIG. 2A is a schematic diagram of a detector array containing three individual detectors. FIG. 2B is a schematic diagram of the reaction region of a single detector. FIG. 2C is a schematic diagram of the chamber region of a detector array;

FIG. 3 is a schematic diagram of a reversible two-step detector;

FIG. 4 is a schematic diagram of another reversible two-step detector; and

FIG. 5 is a schematic diagram of a microfluidics device lacking a diluent chamber and mixing channel.

### DETAILED DESCRIPTION OF THE INVENTION AND BEST MODE

This disclosure provides a microfluidic device that is capable of rapidly testing for multiple diseases (or confirmatory testing of the same disease) in parallel and requires substantially less blood than do traditional clinical methods. At these reduced assay volumes, one vacuum tube, the current standard means of drawing blood, contains sufficient blood to run dozens of different tests. The device is particularly effective for diagnosing STDs, like human papilloma virus (HPV), that can present an early detection problem. Advantageously, the device may be used to test for any pathogen or foreign material that stimulates the production of antigen specific antibodies, allowing several strains to be tested for at once.

The microfluidic diagnostic device described herein solves a number of logistical problems in point-of-care testing for STD's and other infectious diseases. The design ensures standardized dilution which can be calibrated to different disease exposure states, removing the need for caregivers to undertake the tedious task of micropipetting samples and reagents into dozens of wells. The device may be able distinguish diseased, vaccinated and no contact states without performing several additional unnecessary dilutions. Elimination of such intermediate steps reduces the risk of technician exposure to contagious diseases. In preferred embodiments, the device can provide a qualitative visual result, thereby removing the need for a photometer or other optical detection device. These improvements make the microfluidic testing device user friendly and cost effective, reduce the need for additional capital investment, and encourage a higher standard of medical care.

Due to the contained chemistry and high surface to volume ratio inside the device this test method has the potential to provide faster results than presently available alternatives. Testing for several diseases during a regular doctor's visit becomes possible, alleviating the problem of patients who do not return for their test results. This device allows for rapid detection of a large number of diseases that otherwise often require outsourced testing. The low cost of manufacturing will make this device ideal for use in low income areas and smaller facilities.

Generally, the operators need only to collect the blood sample, separate the plasma from cellular fraction (e.g., by centrifugation), and deposit an aliquot of blood plasma into the device. Alternatively, the device may contain an in-line filter to separate blood fractions thereby enabling the operator to simply deposit an aliquot of whole blood into the device before centrifugation. Of course, more extensive pre-testing preparation may be performed as required for each particular set of assay conditions.

The blood, plasma, or other sample is driven through the device and into the reaction chamber by capillary action, gas pressure, centrifugation, or any other appropriate means. The device may be configured to yield a qualitative visual response that is read without the assistance of a photometer or other optical detector and, in such case, ideally reports diseased, vaccinated or “no contact” states. No contact refers to an individual who has never encountered the antigen, meaning they have never had the disease nor have they been vaccinated against it. Quantitative results may be obtained through the use of an optical detection system such as a fluorimeter or a spectrophotometer.

In one preferred embodiment, the device is substantially cylindrical and physically sized to make it backwards compatible, fitting into standard blood centrifuges, which are present in nearly all point-of-care locations. The detection assay is ideally based on the Enzyme Linked Immunosorbent Assay (ELISA) type protocol, specifically “microELISA” for small fluid volumes; however, other detection methodologies appropriate to the type of molecule to be detected may be used (e.g., oligonucleotide-based detection methods, etc.).

### Device Operation

The microfluidic device employs fabrication techniques to make microELISA and other micro-reagent testing inexpensive and easy to use in a point-of-care setting. The blood, plasma, or other fluid sample is introduced into the sample reservoir of the microfluidic device via sample port(s), ideally through a safety cap containing a needle that aspirates the sample using capillary force. These ports may advantageously incorporate accessory spill chambers to trap excess fluid and to prevent overflow in the event that the device is improperly loaded. The fluid reservoir is in fluid communication with a series of sample chambers that will hold the samples after loading until the device is centrifuged.

Ideally, the fluid sample is moved from the sample reservoir to the reaction chamber by capillary action. However, if necessary, the fluid sample may be moved by gas pressure or centrifugal forces. Gas pressure may be applied using any appropriate method. For example, the sample port(s) may be adapted to accept a standard Luer syringe which, upon depression by the operator, creates a positive gas pressure forcing the fluid sample into the reaction chamber. Alternatively, the device may be fitted with a cap that contains a pressurized gas (e.g., N₂ and CO₂) chamber which, when affixed to the device, forms a relatively gas-tight seal and causes the pressurized gas to be released into the sample reservoir. The cap may serve the additional purpose of preventing spillage of the fluid sample during handling, thereby further reducing the likelihood of operator exposure. For devices having waste or other chambers downstream of the reaction chamber, overfilling of the reaction chamber may be prevented by the inclusion of a burst valve at the exit of the reaction chamber. This burst valve is designed to rupture under the forces exerted by centrifugation but not under the gas pressure used to load the reaction chamber.

Once the reaction chamber is loaded, the device is typically allowed to incubate for a period of time determined based on the detection methodology. Further processing of the sample and the device also varies based on the detection methodology and the features of the device.

Once fluid sample has been loaded into the device and the molecules to be detected have been allowed to bind to the immobilized capture molecule in the reaction chamber, the device is ideally placed into a standard laboratory centrifuge (e.g., the same centrifuge used for preparing whole blood samples) for the completion of the reaction and detection process.

In one embodiment, each detector contains a chamber holding a washing solution and a chamber holding a developing solution. The washing solution is used to wash the fluid sample and any unbound molecules out of the reaction chamber. The developing solution contains the reagents necessary to yield a calorimetric result that indicates the presence or absence of the molecule to be detected. The washing chamber and the developing chamber may, optionally, be isolated from the reaction chamber by burst valves. In one embodiment, the burst valve isolating the washing chamber is designed to rupture at a lower pressure than the burst valve isolating the developing chamber. Thus, in this configuration, the device must be centrifuged at a first, slower speed to effect washing of the reaction chamber, and then centrifuged at a second, higher speed to initiate the calorimetric reaction. In another embodiment, either with or without burst valves, the washing chamber is connected to the reaction chamber by a shorter channel length then is the developing chamber. Thus, under centrifugation, the washing solution reaches the reaction chamber first. In yet another embodiment, the developing chamber empties directly into the “top” of the washing chamber, again causing the washing solution to reach the reaction chamber first. It is contemplated that, in this embodiment, there will be an insignificant amount of mixing between the washing and developing solutions.

It is further contemplated that, under certain reaction conditions and for certain clinical uses, the fluid sample may require having a pre-determined volume, a diluent chamber filled with a diluent (e.g., a saline solution), and mixing channels. The diluent chambers lie apart from, but may be parallel to, the sample chambers and both sets of chambers are in fluid communication with one or more mixing channel.

When the centrifuge is activated the fluid sample and diluent is forced into the mixing channels where the mixing process begins, diluting the sample to the working concentration. Alternatively, the fluid sample and diluent may be forced together and into the reaction chamber using gas pressure as above.

In one embodiment, mixing continues in the reaction chamber where the one-step microELISA reagents are stored. The diluted sample reacts with the microELISA reagents in this chamber under conditions suitably adjusted to permit immunological reactions. In other embodiments, each detector further comprises additional reagent chambers in fluid contact with the reaction chamber in order that assays other than one-step microELISA may be performed. Optionally, these reagent chambers may be initially isolated from the reaction chamber by burst valves. The burst valves are preferably configured to burst or open at pre-set pressures (centrifugal forces) and may be configured to burst either substantially simultaneously such that reagent chambers are released together, or at different pressures in order to deliver the various reagents in a sequential (i.e., pre-defined) order.

Each reaction chamber on the device contains pathogen-specific reagents which are used to detect any pathogen or pathogen-specific antibodies contained in the biological sample. These reaction chambers may contain a single type of antigen-specific reagent or multiple antigen-specific reagents for a single pathogen. Alternatively, the reaction chamber may contain antigen-specific reagents specific for a plurality of pathogens. The reaction chambers of a single device may contain the same antigen (redundant tests), different antigens for the same pathogen, or antigens for a plurality of pathogens.

In the most preferred embodiments, the pathogen-specific reagents produce a visible color change in the presence of the pathogen or a pathogenic marker or other molecule to be detected. The colorimetric reporter may be read by a photometer or other optical detector for quantitative (or qualitative) results or just visually for a qualitative test.

### Device Design

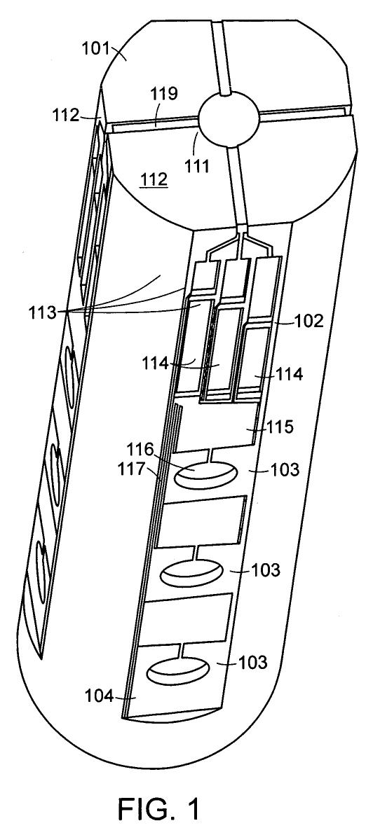

FIG. 1 is a perspective view of a microfluidic device 10 according to the principles of the present disclosure. The device 10 comprises of three regions: the loading region 101, the chamber region 102, and the reaction region 103. A single chamber region 102 and reaction region 103 that are in fluid connection are, together, referred to as a perpendicular detector array 104. The device 10 is physically configured and sized such that it may be inserted into the rotor of a centrifuge. Preferably, the device has substantially the same dimensions as a vacuum tube blood container so that the same centrifuge used to isolate blood plasma at the point-of-care may be used to run the device 10.

The loading region 101 consists of a sample reservoir 111 and a plurality of loading channels 112. It is contemplated that loading region 101 contains a single loading channel 112 providing a fluid connection between sample reservoir 111 and each perpendicular detector array 104; however, a plurality of loading channels 112 may connect to each detector array 104. Further, FIG. 1 illustrates a device 10 having four perpendicular of detector arrays 104; however, the exact number of configuration may be modified to each individual application or user's needs and the maximum number of arrays will depend upon the overall size of the device 10 and the size of each individual detector array 104 and practical manufacturing constraints.

The loading region may optionally contain an overflow chamber (not shown) either on its surface or disposed below, but in fluid contact with, sample reservoir 111. In one embodiment, the overflow chamber is disposed below sample reservoir 111 and is connected via overflow channel 119.

In another embodiment, loading region 101 is covered by a solid surface (not shown) that contains an injector port such that the operator may inject a biological sample (e.g., a blood, plasma, or other fluid sample) through the injector port into sample reservoir 111. The cover may be flat or it may be recessed to provide additional volume capacity to sample reservoir 111.

Optionally, a filter may be disposed between fluid reservoir 111 and loading channels 112 in order to prevent contaminating elements such as blood cells or other large particulate matter from entering detector array 104.

Each detector array 104 comprises of one or more (three are illustrated in FIG. 1) individual detectors.

FIG. 1 illustrates a simple detector configuration. Each detector consists of a sample chamber 113, an optional diluent chamber 114, optional mixing channels 115, and a reaction chamber 116. The sample chamber 113 is in fluid contact with the loading channel 112. Both sample chamber 113 and diluent chamber 114 are in fluid contact with the mixing channels 115 via perpendicular channels 117. The reaction chamber 116 is covered by a transparent or translucent covering such that a colorimetric reaction may be viewed by the operator or measured using an optical detection device.

In one embodiment, loading channels 112 slope down toward sample reservoir 111. In this configuration, the biological sample is loaded into sample reservoir 111 and sample chambers 113 are allowed to fill by capillary action prior to centrifugation. Thus, when placed under centrifugal force, the excess biological sample flows back into sample reservoir 111 and further flows through overflow channel 119 into the overflow chamber below. This configuration advantageously prevents overfilling of sample chambers 113.

The sample chambers 113 of a single detector array may have the same or different volumes. Sample chambers 113 of different volumes are useful to perform serial dilutions of the biological sample against the same detection reagents or when using different detection reagents under well characterized conditions or when serial dilutions are not necessary. Likewise, the volume of diluent chambers 114 may be advantageously modified as desired.

Burst valves 118 may be optionally used to prevent the diluent from escaping diluent chamber 114 prior to use (i.e., during storage) and/or to control the flow of biological sample from sample chamber 113 (i.e., to ensure proper sample volume enters the detector. Likewise, burst valve 118 may be placed between mixing channels 115 and reaction chamber 116 to prevent the escape of reactants from reaction chamber 116 during handling.

Mixing channels 115 are serpentine in configuration and have tight corners to promote turbulent flow which facilitates mixing of the biological sample and the diluent.

The detectors are configured for each individual application and set of reagents to be used.

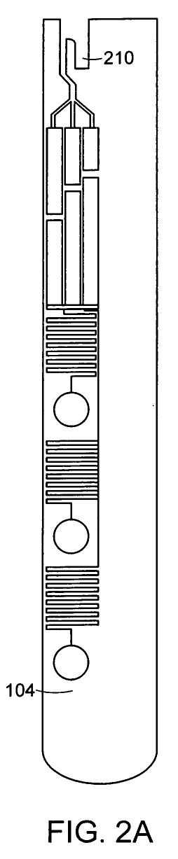

FIGS. 2A-2C are schematic diagrams showing enlargements of the various components of detector array 104 and an individual detector. FIG. 2A illustrates detector array 104 having overflow chamber 210. FIG. 2B illustrates reaction region 103 of a single detector. FIG. 2C illustrates chamber region 102 of a single detector. Loading channel 112 is divided into a plurality of smaller loading channels to conduct the biological sample from loading region 101 (not shown) into individual sample chambers 113.

### Antigen/Antibody-Trap Detectors

In its simplest configuration, the detectors are used to identify antigens of interest in a biological sample. In this embodiment, an antigen-specific antibody is adhered to or otherwise immobilized on the walls of reaction chamber 116. Reaction chamber 116 ideally also contains an antigen-specific antibody that is unbound and detectably labeled, along with any additional reagents required to detect the presence of the antigen of interest. Alternatively, reagents can be stored in other chambers or compartments and added to the reaction chamber as needed by use of flow control devices like the aforementioned burst valves. For example, the unbound antibody may be linked to an enzyme capable of performing a calorimetric reaction as in a one-step ELISA format. Thus, under centrifugal force, the fluid sample that is loaded into sample reservoir 111 is diluted with the diluent contained in diluent chamber 114 and mixed in mixing channels 115. The plasma sample then flows into reaction chamber 116 where the antigen of interest is captured by the immobilized antibody and detected by the unbound antibody by way of a calorimetric reaction (e.g., such as that obtained using horseradish peroxidase and alkaline phosphatase, or any of the other well known enzyme-substrate combinations used in immunoassay systems).

In an alternative embodiment, exposure of the test subject to a pathogen may be determined by detecting the presence of antigen-specific antibodies in the plasma or serum of the test subject. In such cases, the antigen-trap detector described above may be slightly modified in that an pathogenic antigen is immobilized on the wall of reaction chamber 116 which captures the antigen-specific antibodies in the biological sample. These antibodies may then be detected using, for example, an anti-human (or appropriate species depending upon the test subject species) antibody which is ideally linked to a detectable label or to an enzyme capable of catalyzing a colorimetric reaction, as above.

### Two-Step Detectors

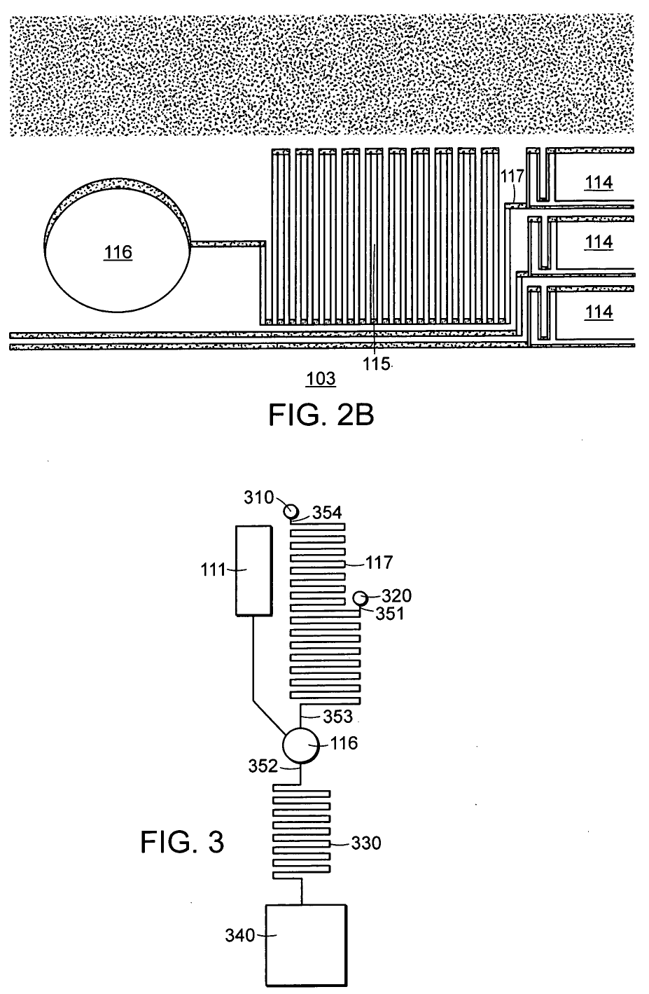

FIG. 3 is a schematic diagram of a single detector that may be used in a “two-step” detection process such as a traditional ELISA assay. In this detector configuration, sample reservoir 111 is in direct fluid contact with reaction chamber 116. Optionally, sample reservoir 111 contains a filter to separate the cellular fraction from the blood plasma/serum or for additional filtration of a plasma/serum sample. Alternatively, sample reservoir 111 lacking a filter is loaded with a plasma or serum sample wherein erythrocytes and other blood cells have been previously removed. In addition, it is contemplated that the device can be configured to permit application of either an undiluted biological sample or a diluted biological sample.

Reaction chamber 116 ideally contains an immobilized capture antibody (if a blood-borne antigen is being assayed) or an immobilized capture antigen (if antigen-specific antibodies are being assayed).

The biological sample fills reaction chamber 116 by capillary action or it may be forced into reaction chamber 116 under pressure (e.g., when sample reservoir 111 is adapted to accept a syringe and positive pressure is applied by depressing the syringe plunger). The binding reaction is permitted to occur in reaction chamber 116 for a sufficient time. The fluid contents are retained in the reaction chamber using a low-pressure burst valve 352 between reaction chamber 116 and waste chamber 340.

The binding reaction is terminated by centrifugation of the device at a first speed. A low pressure burst valve 351 is present between washing chamber 320 and channels 117 that direct the washing buffer into reaction chamber 116. Preferably, the low pressure burst valves 351 and 352 are designed to burst at approximately the same pressure which is the pressure generated by centrifugation at a first speed. Optionally, a fail-safe low pressure burst valve 353 is present to further isolate reaction chamber 116 from the second step reactants (i.e., the washing solution and the developing solution). Burst valves 351, 352, and 353 will rupture at approximately the same time allowing the wash buffer to flow into reaction chamber 116 and then all reaction chamber 116 contents to flow into waste chamber 340. This leaves only the bound elements in the reaction chamber.

Next, the colorimetric reaction is initiated by centrifugation of the device at a second speed that is greater than the first speed. The reagents necessary for the calorimetric reaction, such as a detectably labeled (e.g., enzyme linked) secondary antibody and colorimetric reagents, are contained in the developing solution within developing chamber 310. These reagents are released into reaction chamber 116 by the bursting of high pressure burst valve 354. The reagents are held within reaction chamber 116 because waste chamber 340 and accompanying fluid channels 330 that connect it to reaction chamber 116 are ideally designed to have substantially the same volume as the combined volume of reaction chamber 116 and washing chamber 320. Furthermore, substantially more developing solution may be used than is needed to ensure that the reaction chamber is filled during the colorimetric reaction. As above, the results of the colorimetric reaction may be viewed and/or measured through a transparent or translucent covering on reaction chamber 116.

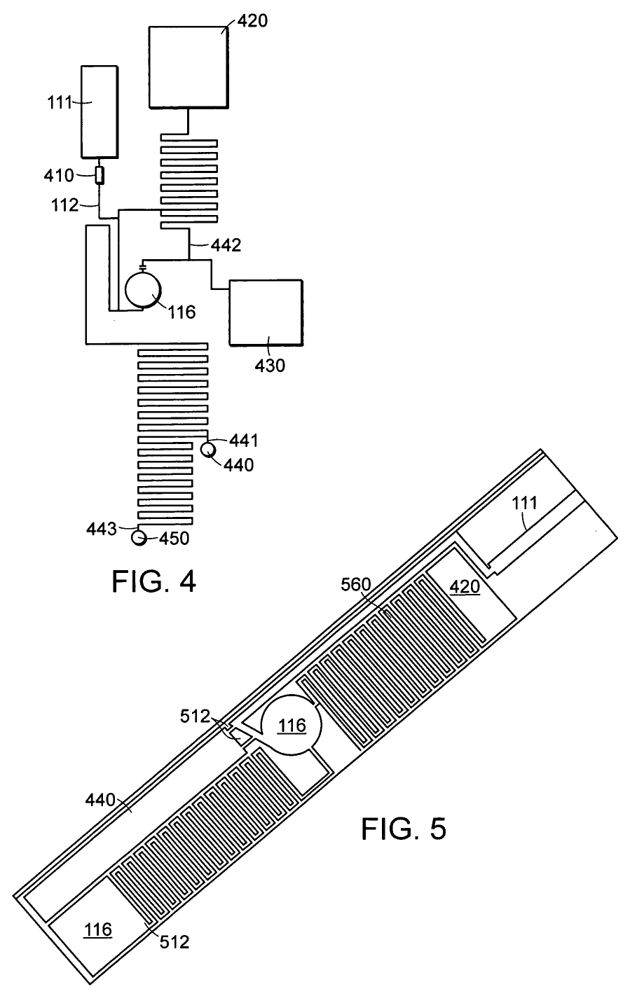

FIG. 5 illustrates another two-step detector configuration. The fluid sample is loaded by capillary action or gas pressure through sample chamber 111 into reaction chamber 116. The fluid sample is allowed to incubate with the immobilized capture molecule. Following incubation, the device is centrifuged, causing burst valves 511 and 512 to rupture allowing the wash solution to flow from wash chamber 440 and the developing solution to flow from the developing chamber 450 through reaction chamber 116 into waste chamber 420. By virtue of the shorter channel length, the wash buffer reaches reaction chamber 116 before the developing solution. The volume of waste chamber 420 and associated channel 560 is substantially the same as the volume of wash buffer and reaction chamber 116. Thus, the developing solution is retained in reaction chamber 116 because waste chamber 420 and channel 560 is full of reaction and wash solutions.

### Reversible Two-Step Detectors

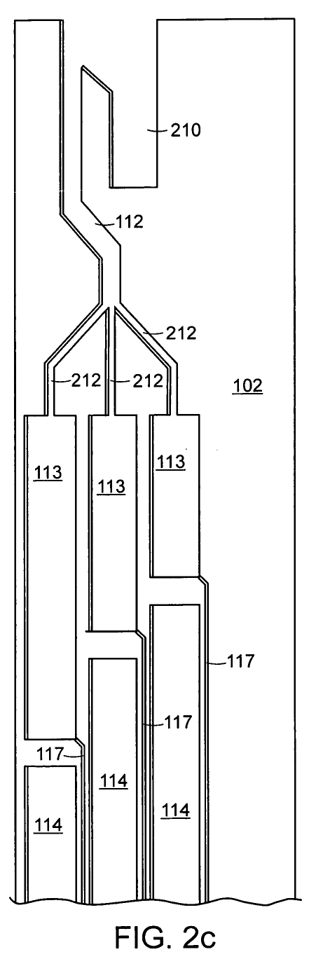

FIG. 4 is a schematic diagram of a reversible two-step detector that is configured to accept biological samples that require filtration prior to ELISA detection (e.g., whole blood). The biological sample is loaded into sample reservoir 111 which is sealed and the device centrifuged at a first speed sufficient to filter the sample through filter 410, with the filtrate (e.g., serum) passing into reaction chamber 116. Optionally, loading channel 112 is in fluid contact with either waste chamber 420 or waste chamber 430 to accommodate excess filtrate. Likewise, reaction chamber 116 may be in fluid contact with the same waste chamber 420 (not shown) or a different waste chamber 430. The binding reaction within reaction chamber 116 is allowed to proceed for an appropriate period of time.

The device is next inverted in the centrifuge (centrifugal force applied in an upward direction in FIG. 4) and centrifuged at a second speed. The second speed is greater than the first and results in the release of wash buffer contained within wash chamber 440 through burst valve 441 that is designed to rupture at the centripetal force generated by the second speed. The wash buffer flows through reaction chamber 116 and, along with the unbound reagents, is captured in one or both of waste chambers 420 and 430. Optionally, a second burst valve 442 is placed between reaction chamber 116 and one of the waste chambers 420 to ensure that the first waste chamber 430 is filled. This may improve the fluid handling characteristics of the device. The second burst valve 442 is designed to rupture at the second speed or above. The second speed is significantly greater than the first speed in order that burst valve 441 remains intact during the filtration process.

The device is further centrifuged at a third speed that is significantly greater than the second speed. The third speed ruptures burst valve 443, releasing the developing solution from developing chamber 450 into reaction chamber 116 in order that the calorimetric reaction occurs. In the event that first waste chamber 430 is not full and burst valve 442 has not ruptured, the volume of the developing solution and the third speed will be sufficient to do so. The principles of the developing solution and calorimetric detection are the same as described above.

### Other Detector Configurations

It is recognized that the exact configuration of the chambers and channels may be modified for the particular needs of each individual assay without departing for the scope and spirit of this disclosure. For example, the immobilized capture molecule may be present in mixing channels 115, obviating the need for reaction chamber 116, but necessitating one or more waste chambers 420. Likewise, the devices of this invention may be configured for detection assays requiring more than two steps. Such device configurations utilize additional reactant chambers in addition to washing chamber 440 and developing chamber 450 described herein. The timing of release of the contents from these additional chambers may be controlled by any appropriate means including the additional burst valves as described herein.

### Alternative Methods for Detection

Although this disclosure has been exemplified by the use of an ELISA-based assay to detect the molecule of interest in the fluid sample, any appropriate detection assay may be used.

Florescence in situ Hybridization (FISH) may be used to detect and identify microorganisms by labeling chromosomes or genes with fluorescently labeled DNA probes that are complimentary to segments of the target organisms' genome. In this embodiment the organism to be detected is collected with a swab, or capillary tube if present in the blood stream, and moved into the reaction chamber. A reactant fluid containing the labeled DNA probes is present in (or introduced into) the reaction chamber. The device is incubated under conditions that allow the probes to penetrate the organism and bind to the target sites (nucleic acids of interest). The device is then centrifuged such that the reaction chamber is emptied of excess fluid and, preferably, the reaction chamber is washed with a washing buffer as described above. Microorganisms are retained in the reaction chamber for later detection either by the use of a filter near the exit channel of the reaction chamber (i.e., that connects the reaction chamber to the waste chamber), or by making the exit channel sufficiently narrow such that the microorganisms cannot pass. If the target organism is present then the DNA probes will remain trapped with in them in the reaction chamber, if they are not there will be few or no DNA probes present in the reaction chamber when the test is complete.

Oligonucleotide ligation reactions may be used to detect nucleic acids in the fluid sample. The reaction chamber contains immobilized capture oligonucleotides that are complimentary to the sequences of interest. The fluid sample is introduced into the reaction chamber and complementary strands will bind to the capture oligonucleotides causing a change in the optical properties of the reaction chamber. Because of the small size of the probes and recent developments in microfabrication and surface treatment thousands of different segments could be probed at the same time using optical detectors. Future technologies may eliminate the need for an optical detector.

EIA could also be preformed by using secondary antibodies which are bound to gold colloids, quantum dots or other visible markers removing the need for developing solutions.

### Device Manufacture

Compression molding of polymers in photolithographically defined micromolds may advantageously be used to form the microfluidic channels on the device. Polymer embossing and micromolding are techniques that enable the fabrication of several hundreds or thousands of inexpensive, disposable parts from one silicon master. The technique enables tight tolerances and high quality control. Molding of engineering polymer resins are useful techniques for mass production, while soft lithography is suitable for rapid prototype development and testing.

Without delving unnecessarily into well known fabrication techniques, it is noted that using transparency masks, it is possible to obtain resolution as low as 20 μm, and features of 50 μm and larger are easily reproduced (Whitesides et al., Annu. Rev. Biomed. Eng. 3: 335-373, 2001). The desired features are first printed on a high resolution transparency. SU-8, a negative photoresist, is spun onto a silcon wafer. The transparency is then placed over the photoresist and exposed to UV light. The UV light causes the negative photoresist to harden while areas not exposed to UV light may be washed away. The image on the transparency is transferred to the SU-8 layer on the silicon wafer. Polydimethyl siloxane (PDMS) and a crosslinking agent are mixed and poured into the micromold. After curing, the pattern of microchannels and reservoirs are transferred to the new media.

All publications and patents cited in this specification are herein incorporated by reference as if each individual publication or patent were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A non-radial cylindrical microfluidic device for analyzing the presence or absence of a molecule to be detected in a fluid sample, said device comprising:
a sample reservoir having a sample input port in fluid connection with at least one detector array,
wherein said detector array comprises at least one detector comprising a reaction chamber having an immobilized capture molecule, and
a reagent capable of undergoing a colorimetric reaction or displaying an optically detectable signal and capable of reacting with said molecule to be detected.

2. The device of claim 1, wherein said device further comprises a diluent chamber and a mixing channel.

3. The device of claim 2, wherein said diluent chamber is isolated from, but capable of fluid communication with, said mixing channel by a first burst valve.

4. The device of claim 1, wherein said at least one detector further comprises a sample chamber having a pre-defined capacity.

5. The device of claim 1, wherein said fluid is selected from the group consisting of whole blood, blood serum, blood plasma, seminal fluid, prostatic fluid, saliva, urine, and spinal fluid.

6. The device of claim 1, wherein said reagent is further capable of binding to said molecule to be detected.

7. The device of claim 1, wherein said immobilized capture molecule is an antigen and said molecule to be detected is an antibody or other soluble binding protein.

8. The device of claim 7, wherein said antigen is a microorganism-specific antigen and said molecule to be detected is an antibody.

9. The device of claim 8, wherein said antigen is an antigen specific for a microorganism selected from the group consisting Chlamydia spp., Gonorrhea spp., human papillomavirus, and human immunodeficiency virus.

10. The device of claim 9, wherein said reagent capable of undergoing a colorimetric reaction is a detectably labeled antibody capable of binding antibodies.

11. The device of claim 10, wherein said detectably labeled antibody is an enzyme-linked antibody and said device further comprises a substrate for said enzyme-linked antibody which, when acted upon by said enzyme, produces an optically detectable response.

12. The device of claim 1, wherein said immobilized capture molecule is an antibody specific for an antigen.

13. The device of claim 12, wherein said antigen is a microorganism-specific antigen and said molecule to be detected is an antibody.

14. The device of claim 13, wherein said antigen is an antigen specific for a microorganism selected from the group consisting Chlamydia spp., Gonorrhea spp., human Papillomavirus, herpes simplex virus, hepatitis B, syphilis, trichomononiasis, bacterial vaganosis and human immunodeficiency virus.

15. The device of claim 14, wherein said reagent capable of undergoing a colorimetric reaction is a detectably labeled antibody capable of binding said antigen.

16. The device of claim 15, wherein said detectably labeled antibody is an enzyme-linked antibody and said device further comprises a colorimetric substrate for said enzyme-linked antibody.

17. The device of claim 1, wherein said colorimetric reaction or optically detectable signal is capable of being viewed by an operator.

18. The device of claim 1, wherein said colorimetric reaction or optically detectable signal is capable of being detected or measuring using an optical sensing device.

19. The device of claim 1, wherein said device further comprises a wash chamber comprising wash buffer and a developing chamber comprising said reagents, wherein said wash chamber is isolated from said reaction chamber by a second burst valve and said developing chamber is isolated from said reaction chamber by a third burst valve, wherein said first burst valve is designed to rupture at a substantially lower pressure than said second burst valve and said second burst valve is designed to rupture at substantially lower pressure than said third burst valve.

20. The device of claim 19, wherein said pressure differences are produced by different centrifugal forces.

21. A method for detecting a molecule in a fluid sample comprising introducing said sample into the device of claim 1, centrifuging said device, detecting the presence or absence of said colorimetric response, and relating the presence or absence of said calorimetric response to the presence or absence of said molecule to be detected.

22. A diagnostic kit for use in detecting a molecule associated with a disease state comprising the device of claim 1 and instructions for its use.
